(19) United States
(12) Patent Application Publication
(10) Pub. No.: US 2026/0325095 A1
(43) Pub. Date: Oct. 22, 2026

(54) RADIOGRAPHIC IMAGE PROCESSING APPARATUS, RADIOGRAPHIC IMAGING SYSTEM, AND NON-TRANSITORY COMPUTER-READABLE RECORDING MEDIUM

(71) Applicant: Konica Minolta, Inc., Tokyo (JP)

(72) Inventors: Kojiro ONO, Chiba (JP); Kentaro OKAMOTO, Tokyo (JP); Ryoichi WATANABE, Tokyo (JP)

(21) Appl. No.: 19/644,380

(22) Filed: Apr. 10, 2026

(30) Foreign Application Priority Data
Apr. 17, 2025 (JP) 2025-068172

(51) Int. Cl. A61B 6/00; G16H 50/20

(52) U.S. Cl. CPC A61B 6/5217; A61B 6/4405; G16H 50/20

(57) ABSTRACT

Provided is a radiographic image processing apparatus that includes a hardware processor. The hardware processor determines information for categorizing a pathological condition in a region of interest in, among a plurality of frames of a dynamic image, a specific frame based on first information, which is based on a signal value of the region of interest of the specific frame, and second information, which is based on a temporal change amount of the signal value in the region of interest of the dynamic image.

### CROSS REFERENCE TO RELATED APPLICATIONS

The entire disclosure of Japanese patent Application No. 2025 -068172, filed on Apr. 17, 2025, is incorporated herein by reference in its entirety.

### BACKGROUND

### 1. Technological Field

The present invention relates to a radiographic image processing apparatus, a radiographic imaging system, and a non-transitory computer-readable recording medium.

### 2. Description of the Related Art

In the related art, there is known a radiographic imaging system capable of capturing a dynamic image of a subject. For example, WO 2012/026146 discloses a system capable of capturing a dynamic image, which has a configuration in which analysis is performed for each of small regions associated with each other among a plurality of frame images to create a histogram (temporal changes in signal values) of values of a ratio of the inspiratory feature quantity to the expiratory feature quantity.

In addition, at present, the status of the chest of post-operative patients in intensive care is generally observed by simple X-ray (still image) imaging. However, since it is difficult to grasp, with a still image, the timing of breathing of a post-operative patient who has difficulty in spontaneous breathing, highly reliable diagnosis cannot be performed and consequently there is a risk that appropriate treatment cannot be performed.

On the other hand, in a system capable of capturing a dynamic image, a plurality of frames is acquired from the dynamic image, it is possible to easily obtain a frame at the timing of breathing of a subject. That is, a system capable of capturing a dynamic image is expected to be utilized as a system that makes it possible to perform appropriate treatment for post-operative patients in intensive care.

### SUMMARY

However, there are cases where it may be difficult to accurately grasp the pathological condition in a chest examination only based on temporal changes in signal values. For example, since there is no large difference in temporal changes in signal values between emphysema and atelectasis, it is difficult to distinguish the pathological conditions from each other. That is, the configuration described in WO 2012/026146 has room for improvement as a configuration for supporting diagnosis of a radiographic image.

An object of the present invention is to provide a radiographic image processing apparatus, a radiographic imaging system, and a non-transitory computer-readable recording medium each capable of grasping a pathological condition in a chest examination accurately.

In order to achieve at least one of the above-described objects, a radiographic image processing apparatus reflecting one aspect of the present invention is a radiographic image processing apparatus that processes a dynamic image including a plurality of frames obtained by a dynamic image capturing apparatus related to radiographic imaging.

The radiographic image processing apparatus includes a hardware processor that sets a region of interest in, among the plurality of frames of the dynamic image, a specific frame in which a predetermined status in a chest of a subject has been specified.

The hardware processor determines information for categorizing a pathological condition in the region of interest based on first information, which is based on a signal value of the region of interest of the specific frame, and second information, which is based on a temporal change amount of the signal value in the region of interest of the dynamic image.

In order to realize at least one of the above-described objects, a radiographic imaging system reflecting one aspect of the present invention is a radiographic imaging system that includes: a dynamic image capturing apparatus; and a radiographic image processing apparatus that processes a dynamic image including a plurality of frames obtained by the dynamic image capturing apparatus.

The radiographic image processing apparatus includes a hardware processor that sets a region of interest in, among the plurality of frames of the dynamic image, a specific frame in which a predetermined status in a chest of a subject has been specified.

The hardware processor determines information for categorizing a pathological condition in the region of interest based on first information, which is based on a signal value of the region of interest of the specific frame, and second information, which is based on a temporal change amount of the signal value in the region of interest of the dynamic image.

In order to realize at least one of the above-described objects, a program reflecting an aspect of the present invention is a program stored in a non-transitory computer-readable recording medium storing a program for a radiographic image processing apparatus to process a dynamic image including a plurality of frames acquired by a dynamic image capturing apparatus related to radiographic imaging.

The non-transitory computer-readable recording medium stores the program that causes a computer to execute:
setting a region of interest in, among the plurality of images of the dynamic image, a specific frame in which a predetermined status in a chest of a subject has been specified; and
determining information for categorizing a pathological condition in the region of interest based on first information, which is based on a signal value of the region of interest of the specific frame, and second information, which is based on a temporal change amount of the signal value in the region of interest of the dynamic image.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating a radiographic imaging system according to an embodiment of the present invention;

FIG. 2 is a block diagram illustrating the configuration of a main part of a control system according to the present embodiment;

FIG. 3 is a block diagram illustrating an imaging control section;

FIG. 4 is a diagram illustrating a two-dimensional graph in which pathological conditions disease have been categorized;

FIG. 5 is a flowchart illustrating exemplary operations of determination control by the imaging control section; and

FIG. 6 is a diagram illustrating the radiographic imaging system according to a variation.

### DETAILED DESCRIPTION OF EMBODIMENTS

Hereinafter, one or more embodiments of the present invention will be described with reference to the drawings. However, the scope of the invention is not limited to the disclosed embodiments.

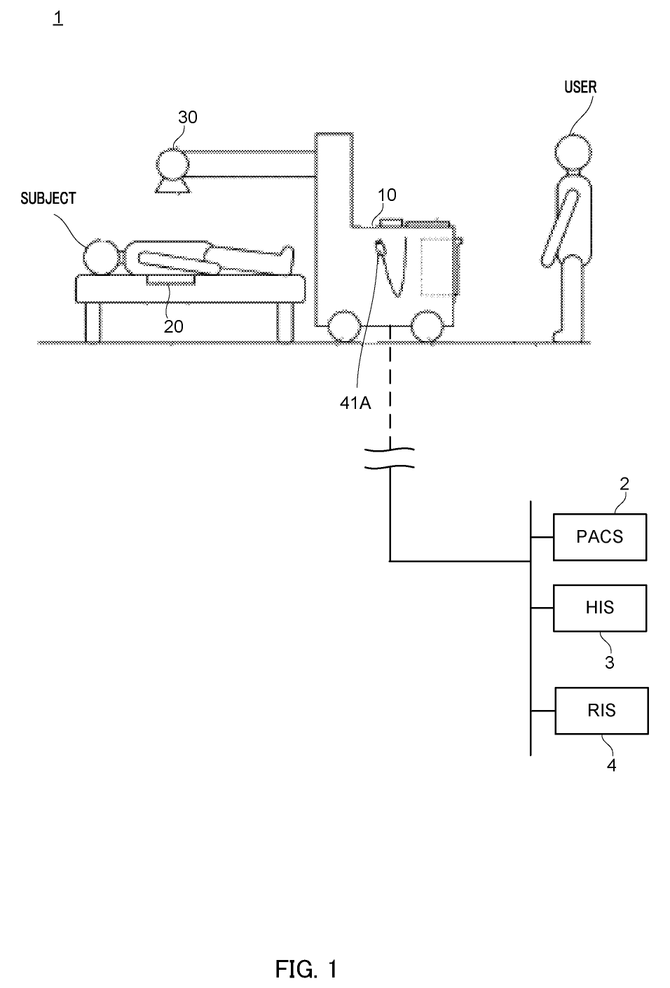

Hereinafter, an embodiment of the present invention will be described in detail with reference to the accompanying drawings. FIG. 1 is a diagram illustrating a radiographic imaging system according to an embodiment of the present invention.

As illustrated in FIG. 1, a radiographic imaging system 1 according to the present embodiment is capable of capturing still images and dynamic images, and is a visiting system for visiting a subject (patient), who has difficulty in movement, and capturing a radiographic image. Note that, the radiographic imaging system 1 is applicable to, for example, a hospital room, an intensive care unit (ICU), an operating room, and the like.

Capturing a dynamic image here refers to obtaining a plurality of frame images by repeatedly irradiating a subject with pulsed radiation (for example, X-rays) at a predetermined frame rate (pulse irradiation). In addition, the dynamic image refers to a series of frame images obtained by capturing a dynamic image. In addition, it is also possible to perform dynamic analysis on a captured dynamic image. The dynamic analysis refers to analysis processing performed on a dynamic image, and includes, in addition to processing of analyzing the motion of the subject on the basis of the dynamic image, processing of analyzing the dynamic image to highlight or attenuate (remove) a predetermined structure.

The radiographic imaging system 1 includes a dynamic image capturing apparatus constituted by a main body 10, at least one flat panel detector (FPD) 20, and a radiation irradiation apparatus 30. The dynamic image capturing apparatus including the main body 10 and the radiation irradiation apparatus 30 is configured as a movable medical cart as an example and is an apparatus suitable for visiting a subject who has difficulty in moving to another room. In addition, the medical cart is suitable for dynamic imaging of a subject who has difficulty in moving from the ICU to another room.

In addition, the radiographic imaging system 1 may also include external systems connected to the main body 10 via a communication network. Examples of the external systems include picture archiving and communication systems (PACS) 2, and hospital information systems (HIS) 3, radiology information systems (RIS) 4.

In the communication network including the radiographic imaging system 1 (the main body 10), the PACS 2, the HIS 3, and the RIS 4, information is transmitted and received, for example, in accordance with a digital image and communications in medicine (DICOM) standard.

The FPD 20 is a portable radiation detector that is compatible with capturing of still images and dynamic images. The FPD 20 includes, for example, radiation detection elements two-dimensionally arranged on a glass substrate. The radiation detection element is constituted by a semiconductor image sensor such as a photodiode. The radiation detection element detects radiation, which has been irradiated from the radiation irradiation apparatus 30 (radiation source) and has passed through at least a subject, according to the intensity thereof, converts the detected radiation into an electrical signal, and accumulates the electrical signal. For example, a switching section such as a thin film transistor (TFT) is connected to each radiation detection element. The switching section controls the accumulation and reading of electrical signals, and image data is acquired.

Note that, the FPD 20 may be of an indirect conversion type which converts radiation into an electrical signal by a photoelectric conversion element via a scintillator, or may be of a direct conversion type which directly converts radiation into an electrical signal.

In addition, the FPD 20 is connected to the main body 10 of the radiographic imaging system 1 via the communication network, and is configured to perform wired communication or wireless communication with the main body. Specifically, the FPD 20 is configured to receive various control signals from the main body 10 and transmit generated image data to the main body 10 via a communication cable.

In addition, the main body 10 is provided with a housing section (not illustrated) capable of housing the FPD 20. The main body 10 is conveyed to a hospital room in a state in which the FPD 20 is housed in the housing section.

The radiation irradiation apparatus 30 includes a synchronization signal output section (not illustrated), a generator (not illustrated), a radiation source (not illustrated), and the like.

The synchronization signal output section outputs a pulsed synchronization signal to each of the generator and the FPD 20 in response to an operation of an exposure switch 41A, which will be described later. When capturing still images, a synchronization signal is transmitted only once per still image, while when capturing dynamic images, a synchronization signal of the same cycle is repeatedly transmitted a plurality of times.

The generator is configured to be capable of applying, to the radiation source, a voltage corresponding to radiation irradiation conditions set in advance (a tube voltage, a tube current, an irradiation time, and the like) each time a synchronization signal is inputted from the synchronization signal output section.

The radiation source (tube) includes a rotating anode, a filament, and the like. The rotating anode is constituted by, in addition to a target, a rotor or an anode rotor that rotates the anode, an anode shaft, a bearing (not illustrated), and the like. The anode rotor rotates the target at a high speed by the principle of an induction motor. The filament is provided on a cathode sleeve disposed at a position at which the filament faces the target. When a voltage is applied from the generator, the filament emits an electron beam corresponding to the voltage toward the rotating anode, and the rotating anode (anode rotor) generates a dose of radiation corresponding to the intensity of the electron beam.

In addition, the radiation source is rotated by a rotation shaft extending in the horizontal direction, and can be switched to a state in which an irradiation port of radiation is directed in the horizontal direction (a state in which imaging in a standing position is performed) or a state in which the irradiation port is directed in the vertical direction (a state in which imaging in a lying position is performed).

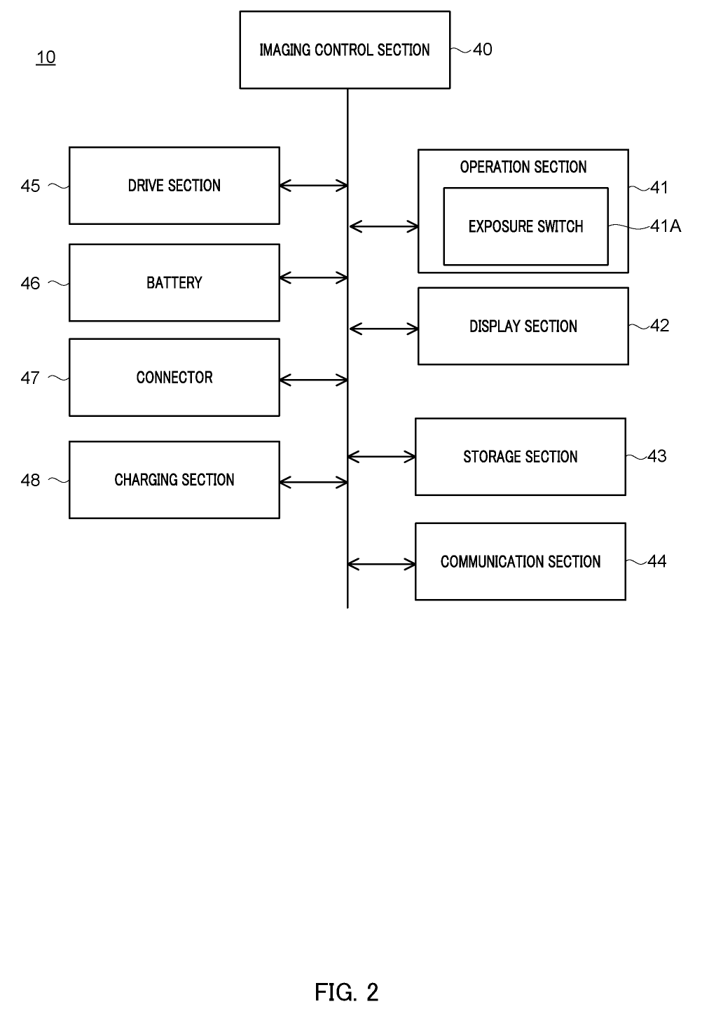

In addition, the main body 10 has a function as a console (radiographic imaging control apparatus). As illustrated in FIG. 2, the main body 10 includes an imaging control section 40, an operation section 41, a display section 42, a storage section 43, a communication section 44, a drive section 45, a battery 46, a connector 47, a charging section 48, and the like.

The imaging control section 40 is housed in the main body 10 and includes a central processing unit (CPU), a read only memory (ROM), a random access memory (RAM), and the like. In the imaging control section 40, the CPU reads a program corresponding to the processing content from the ROM and develops the read program in the RAM. Then, the imaging control section 40 cooperates with the developed program to centrally control the operation of each section.

In addition, the imaging control section 40 determines information for categorizing a pathological condition of a subject based on first information, which is related to a specific frame among a plurality of frames of a dynamic image, and second information, which is based on a temporal change amount of a predetermined signal value of the dynamic image. The determination control of the above information by the imaging control section 40 will be described later.

The operation section 41 includes a touch screen or the like in which transparent electrodes are arranged in a lattice shape so as to cover the surface of the display section 42. The touch screen detects a position pressed with a finger, a touch pen, or the like, and inputs the position information as operation information to the imaging control section 40.

In addition, the operation section 41 includes an exposure switch 41A for operating the radiation irradiation apparatus 30. The exposure switch 41A is a switch for the user to instruct radiation irradiation by the radiation irradiation apparatus 30.

The display section 42 is constituted by a monitor such as a liquid crystal display (LCD) or a cathode ray tube (CRT). The display section 42 performs display in accordance with an instruction of a display signal inputted from the imaging control section 40.

The storage section 43 is constituted by a non-volatile semiconductor memory, a hard disk, or the like. The storage section 43 stores various programs executed by the imaging control section 40, parameters necessary for execution of processing by the programs, or data such as processing results.

In addition, the storage section 43 is provided with a primary storage region (not illustrated) for temporarily storing the radiographic image transferred from the FPD 20. Further, the storage section 43 is provided with an image storage region (not illustrated) for storing the radiographic image transferred from the FPD 20 in association with supplementary information for a certain period of time.

The communication section 44 transmits and receives data to and from the FPD 20 by wired communication or wireless communication. In addition, the communication section 44 transmits and receives data to and from the external apparatuses, such as the RIS and the PACS, via the communication network.

The drive section 45 is a circuit that drives the tube of the radiation source of the radiation irradiation apparatus. The drive section 45 and the radiation source are connected to each other via a cable.

The battery 46 supplies electric power to each section of the main body 10, and the radiation source. The battery 46 can be charged from the outside via an AC cable.

The connector 47 is provided inside the housing section and is electrically connected to the FPD 20 housed in the housing section.

The charging section 48 charges the FPD 20, which is connected thereto via the connector 47, with the electric power supplied from the battery 46 on the basis of the control by the imaging control section 40.

Next, the determination control by the imaging control section 40 will be described.

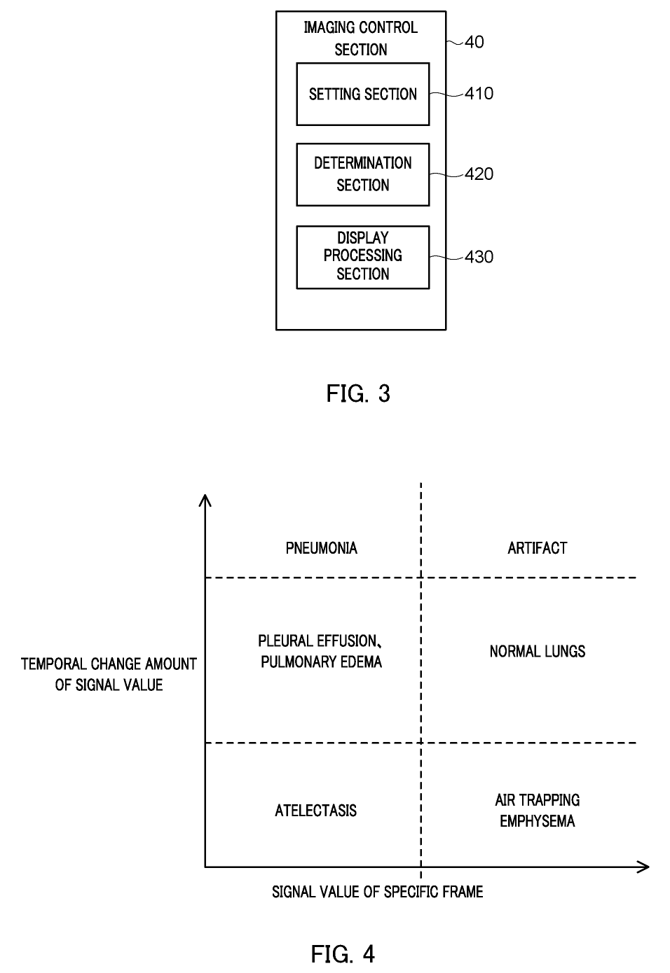

The imaging control section 40 processes a dynamic image including a plurality of frames obtained by the radiographic imaging apparatus to determine information for categorizing a pathological condition of a subject as described above. The dynamic image obtained here is a dynamic image obtained by dynamic imaging of the chest of the subject. Specifically, the dynamic image is an image including an image of the chest of the subject (an image in which an image of the chest has been captured), for example, an image including an image of the lung field of the subject. As illustrated in FIG. 3, the imaging control section 40 includes a setting section 410, a determination section 420, and a display processing section 430.

The setting section 410 sets a region of interest in, among a plurality of frames of a dynamic image, a specific frame in which a predetermined status in the chest of the subject has been specified. The predetermined status is a respiratory status indicating a status in which the subject is breathing, and may be, for example, among respiratory statuses, a status of the maximum inspiration during quiet breathing. In addition, the predetermined status may be a status other than a respiratory status, such as a status in which the lung field is moving.

For example, the specific frame may be specified by the imaging control section 40 based on a change in a signal value (for example, a density value of an image) when the subject is breathing. Specifically, when the subject is breathing, a waveform that oscillates between expiration and inspiration is obtained, and thus, a frame at a timing corresponding to inspiration (for example, a timing of the maximum inspiration during quiet breathing) is specified as the specific frame.

The region of interest is, in an imaging portion of the subject, a region (region of interest) from which data (signal value) for checking a pathological condition is acquired. For example, the region of interest may be a region set by a setting operation of the user or may be a region set by automatic setting of the imaging control section 40. For example, the region of interest can be set to a region including the lung field of the chest.

The setting operation of the user is, for example, an operation in which the user sets a region of interest in a specific frame via the operation section 41 or the like. The setting operation of the user may be performed by the user checking a chest image (radiographic image) of a subject in advance or may be performed after the user checks a specific frame after a radiographic image is captured. In addition, in a case in which the user checks a specific frame after a radiographic image is captured, the specific frame may be presented to the user, for example, displayed on the display section 42, before the setting by the setting section 410.

For example, the region set by the automatic setting of the radiographic imaging control apparatus may be a region set in advance in a specific frame or may be a region set based on the color value of the image in the specific frame.

The color value of the image in the specific frame is, for example, a value based on the signal value of the radiation transmission intensity. For example, in a range in which the signal value of the radiation transmission intensity is relatively high (hereinafter, referred to as the first range), it means that a relatively large amount of radiation is transmitted through the object. In a range in which the signal value of the radiation transmission intensity is relatively low (hereinafter, referred to as the second range), it means that a relatively large amount of radiation has been absorbed by the object. In an image, for example, the values of the first range are represented by darker color values than the values of the second range. Signal values in a radiographic image of the chest are distributed so as to be different mainly among the lungs, the heart and the soft tissue, and the bone. The lungs are mainly filled with air, and therefore absorb little radiation. For this reason, since the amount of radiation detected by the detector is relatively large in the lung portion, the signal value of the radiation transmission intensity is relatively high. Since the heart and the soft tissue absorb radiation to some extent, radiation is less transmitted therethrough as compared with the lungs. For this reason, since the amount of radiation detected by the detector in the heart and the soft tissue is smaller than that in the lungs, the signal value of the radiation transmission intensity is medium (for example, with a color brighter than that of the lung portion). The bone contains a large amount of calcium, and therefore absorbs a large amount of radiation. For this reason, since the amount of radiation detected by the detector is relatively small in the bone portion, the signal value of the radiation transmission intensity becomes a relatively low value (for example, with a color brighter than that of the heart and the soft tissue). For example, in a 12-bit digital radiographic image, the signal values of the lung portion are in a range of 3000 to 4000, the signal values of the heart and the soft tissue are in a range of 1000 to 2500, and the signal values of the bone portion are in a range of 0 to 500. In addition, the signal value may be an absolute value. For example, the setting section 410 sets, based on the color values of an image in a specific frame, a region, in which color values in a range of medium signal values (for example, the second range described above) or in a range of relatively high signal values (for example, the first range described above) are present at least in a predetermined value, as a region of interest.

In addition, the region set by the automatic setting of the radiographic imaging control apparatus may be, for example, a region set by using artificial intelligence. The region set by using artificial intelligence is, for example, a region set by detecting a region in which a pathological condition is suspected by the artificial intelligence. As a detection method by artificial intelligence, for example, a method using artificial intelligence known in the art or the like is applicable.

The determination section 420 determines information for categorizing a pathological condition in a region of interest based on the first information and the second information that have been described above.

The first information is information based on the signal value of a region of interest of a specific frame. For example, since air is sent into the lungs of the subject during inspiration, the signal value of the radiation transmission intensity becomes high, and the portion of the lungs in the radiographic image is displayed in a color corresponding to the first range. However, in the case of a subject having some disease, air is not sent into the lungs even during inspiration, and thus, the signal value of the radiation transmission intensity becomes low, and the portion of the lungs in the radiographic image is displayed in a color corresponding to the second range. For this reason, it is possible to classify a pathological condition by acquiring information on the magnitude of the signal value in the first information.

For example, in the case of a subject having normal lungs, air is normally sent into the lungs of the subject, and thus, the signal value of the radiation transmission intensity becomes high, and the portion of the lungs in the radiographic image is displayed in a color corresponding to the first range. In addition, in the case of a subject with emphysema, air is likely to remain in the lungs of the subject, and thus, the signal value of the radiation transmission intensity becomes high, and the portion of the lungs in the radiographic image is displayed in a color corresponding to the first range. In addition, in the case of a subject with air trapping, air is left in the lungs, and thus, the signal value of the radiation transmission intensity becomes high, and the portion of the lungs in the radiographic image is displayed in a color corresponding to the first range.

In addition, in the case of a subject with atelectasis, the air included in the lungs of the subject decreases, and thus, the signal value of the radiation transmission intensity becomes low, and the portion of the lungs in the radiographic image is displayed in a color corresponding to the second range. In addition, in the case of a subject with pleural effusion or pulmonary edema, liquid is accumulated outside or inside the lungs, and thus, the signal value of the radiation transmission intensity becomes low, and the portion of the lungs in the radiographic image is displayed in a color corresponding to the second range due to the influence of the liquid. In addition, in the case of a subject with pneumonia, secretion or pus spreads in the portion of the air in the lungs, and thus, the signal value of the radiation transmission intensity becomes low, and the portion of the lungs in the radiographic image is displayed in a color corresponding to the second range.

For example, the determination section 420 determines, based on the first information, whether the pathological condition of the subject is classified into the first range, in which the signal value is within the predetermined range, or the second range, in which the signal value is in a range other than the predetermined range.

The predetermined range is, for example, a range in which the signal value is equal to or greater than the predetermined value. That is, the first range is a range in which the signal value is equal to or greater than the predetermined value, and the second range is a range in which the signal value is less than the predetermined value.

That is, it is possible to classify, based on the first information, whether the subject may have a pathological condition for which a radiographic image is displayed in a color corresponding to the second range or may have a pathological condition for which a radiographic image is displayed in a color corresponding to the first range.

The second information is information based on a temporal change amount of the signal value in a region of interest of a dynamic image, and may be, for example, a change rate of the signal value of the maximum expiration with respect to the signal value of the maximum inspiration in the dynamic image as a whole.

The determination section 420 determines, based on the second information, information for categorizing a pathological condition in a region of interest within a range in which the pathological condition of the subject is classified.

For example, it is assumed that a pathological condition is classified into a range in which a radiographic image is displayed in a color corresponding to the second range. In this case, in the case of a subject with atelectasis, the amount of air included in the lungs is small regardless of whether during expiration or during inspiration, and thus, the temporal change amount of the signal value becomes small. In addition, in the case of a subject with pleural effusion or pulmonary edema, a radiographic image is displayed in a color corresponding to the second range due to the influence of liquid, but the amount of air included in the lungs changes between during expiration and during inspiration, and thus, the temporal change amount of the signal value becomes larger than that of a subject with atelectasis. In addition, in the case of a subject with pneumonia, a radiographic image is displayed in a color corresponding to the second range, but air is sent into the lungs during expiration and during inspiration, and thus, the temporal change amount of the signal value becomes larger than that of a subject with atelectasis, and in some cases, is larger than that of a subject with pleural effusion or pulmonary edema.

For this reason, it is possible to determine, based on the second information, whether the pathological condition of the subject is atelectasis, pleural effusion or pulmonary edema, or pneumonia.

In addition, it is assumed that a pathological condition is classified into a range in which a radiographic image is displayed in a color corresponding to the first range. In this case, in the case of a subject with emphysema or air trapping, air remains in the lungs regardless of whether during expiration or during inspiration, and thus, the temporal change amount of the signal value becomes small. In addition, in the case of a subject with normal lungs, the amount of air included in the lungs changes between during expiration and during inspiration, and thus, the temporal change amount of the signal value becomes large.

For this reason, it is possible to determine, based on the second information, whether the pathological condition of the subject is emphysema or air trapping.

In addition, in a case where a pathological condition has been classified into a range in which a radiographic image is displayed in a color corresponding to the first range and an artifact occurs due to movement of a structure such as the diaphragm, the rib cage, or the heart of a subject, the temporal change amount of the signal value may become large.

Even with such an artifact, it is possible to determine, based on the second information, that the temporal change amount of the signal value becomes large due to the artifact.

As for pathological conditions, for example, it is possible to cause the ranges of the respective pathological conditions to be segmented in the two-dimensional graph illustrated in FIG. 4. That is, the determination section 420 may determine information for categorizing a pathological condition based on a two-dimensional graph constituted by a first axis (the horizontal axis), which indicates signal value information of a specific frame, and a second axis (the vertical axis), which indicates a temporal change amount of a signal value of a dynamic image as a whole.

The display processing section 430 performs processing of outputting, to the display section 42 (display medium), information for categorizing a pathological condition determined by the determination section 420.

The information for categorizing a pathological condition is information indicating the categorization of the pathological condition to the extent that the user can discriminate the type of the pathological condition (the status of the subject). The information for categorizing a pathological condition may be, for example, information of a radiographic image in which the display color is changed for each pathological condition. Specifically, for example, in a dynamic image to be reproduced, the display color of a pathological condition-related portion in a region of interest is changed for each of atelectasis, pleural effusion, pulmonary edema, pneumonia, emphysema, normal lungs, and the like. The difference in display color allows the user to discriminate the type of the pathological condition.

In addition, the information for categorizing a pathological condition may be information indicating the content of the pathological condition. Specifically, for example, text information indicating that “the pathological condition is emphysema” may be displayed as the information for categorizing a pathological condition on the display section 42.

In addition, some pathological conditions are classified into the same range although there is no difference in the temporal change amount of the signal value therebetween. For example, pleural effusion and pulmonary edema are classified into the same range although there is no difference in the temporal change amount of the signal value therebetween. In addition, although pleural effusion and pulmonary edema have been categorized in a different manner from pneumonia in FIG. 4, in some cases, pleural effusion and pulmonary edema have no difference from pneumonia in the temporal change amount of the signal value.

For this reason, for example, text information indicating that “the pathological condition may be pleural effusion, pulmonary edema, or pneumonia” may be displayed as the information for categorizing a pathological condition on the display section 42. That is, the information for categorizing a pathological condition may also include information indicating the possibility of a plurality of pathological conditions.

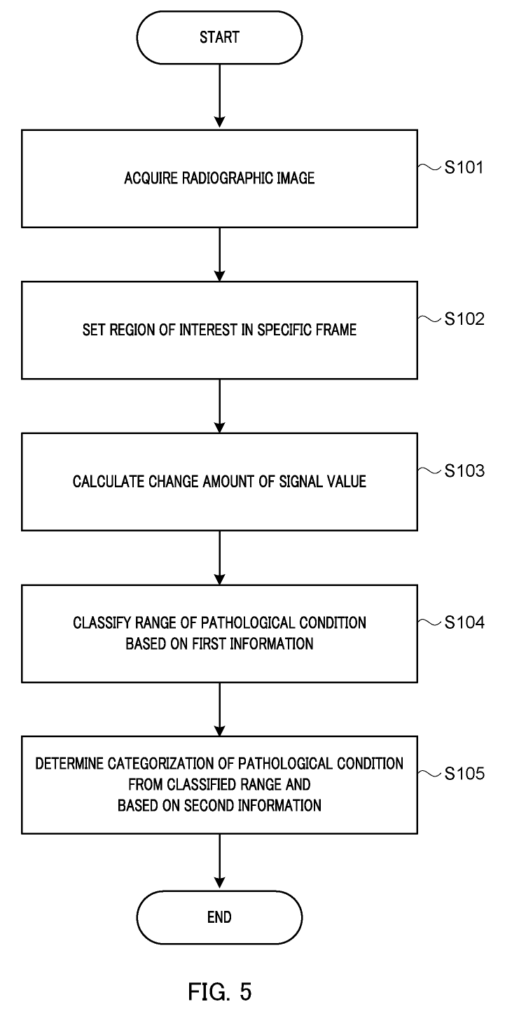

Next, a flow of processing performed by the imaging control section 40 will be described. FIG. 5 is a flowchart illustrating exemplary operations of the determination control by the imaging control section 40. This control is appropriately executed in the radiographic imaging system 1 when the imaging control section 40 acquires a captured radiographic image (dynamic image).

As illustrated in FIG. 5, the imaging control section 40 acquires a radiographic image (step S101). After step S101, the imaging control section 40 sets a region of interest in a specific frame among a plurality of frames of the radiographic image (dynamic image) (step S102).

After step S102, the imaging control section 40 calculates the change amount of the signal value in the region of interest (step S103). After step S103, the imaging control section 40 classifies the range of the pathological condition based on the first information (step S104). After step S104, the imaging control section 40 determines information for categorizing the pathological condition from the classified range and based on the second information (step S105). After step S105, the present control ends.

According to the present embodiment configured as described above, the range of a pathological condition of a subject is classified based the first information, and information for categorizing the pathological condition of the subject is determined from the classified range and based on the second information.

Incidentally, it may be difficult to grasp a pathological condition in a chest examination accurately only based on temporal changes in signal values. For example, emphysema is displayed in a color corresponding to the first range in a radiographic image, whereas atelectasis is displayed in a color corresponding to the second range in a radiographic image. However, in a radiographic image, there is no large difference in temporal changes in signal values of the radiographic image between emphysema and atelectasis. For this reason, it is difficult to grasp a pathological condition in a chest examination only based on temporal changes in signal values.

In addition, normal lungs are displayed in a color corresponding to the first range in a radiographic image, whereas pulmonary edema is displayed in a color corresponding to the second range in a radiographic image. However, in a radiographic image, there is no large difference in temporal changes in signal values of the radiographic image between normal lungs and pulmonary edema. For this reason, it is difficult to grasp a pathological condition in a chest examination only based on temporal changes in signal values.

In this way, it may be difficult to grasp a pathological condition accurately only based on temporal changes in signal values.

In the present embodiment, on the other hand, information for categorizing a pathological condition of a subject is determined from a range, which has been classified based the first information, and based on the second information, and thus, it is possible to grasp a pathological condition in a chest examination accurately. As a result, highly reliable diagnostic support can be performed.

In addition, although it is not possible to distinguish pathological conditions belonging to the same range (for example, pneumonia, pleural effusion, pulmonary edema, atelectasis, and the like in the second range) from each other only based on the signal value of the specific frame, it is possible to easily distinguish the pathological conditions belonging to the same range from each other by adding the temporal change amount of the signal value to the determination.

Incidentally, for post-operative patients in intensive care or the like, there is a demand for grasping pathological conditions of emphysema, atelectasis, pneumonia, pleural effusion, pneumothorax, and the like and taking appropriate therapeutic intervention for each of the pathological conditions. At present, the status of the chest of post-operative patients in intensive care is generally observed by simple X-ray (still image) imaging. Post-operative patients in intensive care have difficulty in spontaneous breathing, and have difficulty in taking an inspiratory position or an expiratory position. For this reason, it is difficult to grasp which timing of breathing a still image obtained by imaging corresponds to.

With such a still image, it is possible to grasp a rough status such as an infiltrative shadow or hyperlucency, but it is not possible to perform highly reliable diagnosis. Further, it is not possible to accurately determine the categorization of the pathological condition related to infiltrative shadow and the categorization of whether the lungs involve hyperlucency or are normal lungs. As described above, in the status observation of the chest based on a captured still image in intensive care, the categorization of a pathological condition such as pulmonary edema, atelectasis, pneumonia, pleural effusion, pneumothorax, or the like cannot be accurately determined. Accordingly, in a case where an appropriate treatment has not been performed, the pathological condition may be further deteriorated.

In the present embodiment, on the other hand, a plurality of frames is acquired by a dynamic image, and thus, it is possible to easily obtain a frame (specific frame) at the timing of breathing of the subject. In addition, the range of a pathological condition is classified based on the specific frame and the first information, and the categorization of the pathological condition is determined based on the classified range and the second information. As a result, it is possible to determine the categorization of a pathological condition accurately and to perform highly reliable diagnosis. Accordingly, it is possible to perform appropriate treatment, and consequently it is possible to suppress deterioration of the pathological condition.

In addition, when diagnosis using computed tomography (CT) can be performed, the categorization of a pathological condition can be accurately determined even by using the related art. However, since there are also patients who have difficulties in moving to the CT room, such as seriously injured patients, there is a possibility that appropriate treatment cannot be performed when such patients are diagnosed based on a captured still image as in the related art. In addition, there is also a case where the CT room is occupied due to reservations, and thus, there is also a possibility that imaging cannot be performed at an appropriate timing.

In the present embodiment, on the other hand, the radiographic imaging system 1 of a mobile type is provided, and thus, a radiographic image can be captured by moving the radiographic imaging system 1 to a patient. As a result, appropriate treatment can be performed, and imaging can be performed at appropriate timing.

Note that, in the above-described embodiment, the specific frame is a frame in which the status of the maximum inspiration during quiet breathing of a subject is specified, but the present invention is not limited thereto, and the specific frame may be a frame in which a status other than the status of the maximum inspiration during quiet breathing is specified, as long as it is the status during inspiration.

In addition, in the above-described embodiment, the radiographic imaging control apparatus (radiographic image processing apparatus) is the radiographic imaging system 1 including the radiation irradiation apparatus 30, but the present invention is not limited thereto. For example, a radiographic imaging control apparatus separate from the radiation irradiation apparatus may be disposed in an operation room different from an imaging room in which the radiation irradiation apparatus is disposed.

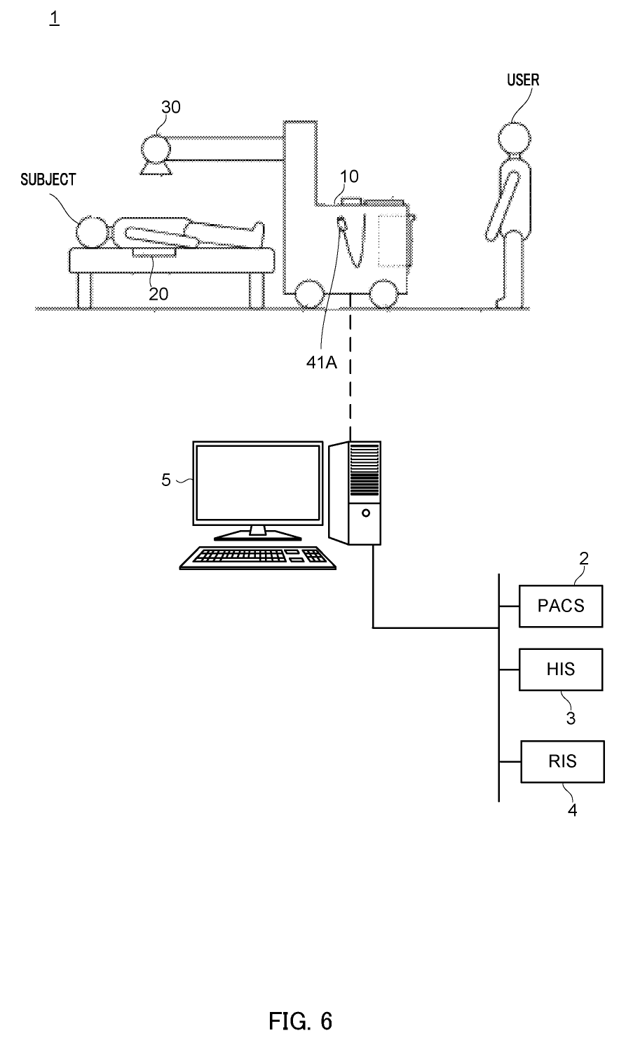

In addition, in the above-described embodiment, the radiographic image processing apparatus is incorporated in the main body 10 (the imaging control section 40), but the present invention is not limited thereto. For example, as illustrated in FIG. 6, a personal computer (PC) 5 which is an external apparatus may be the radiographic image processing apparatus.

The PC 5 includes a CPU, a ROM, a RAM, and the like, and has the same functions as the setting section 410, the determination section 420, the display processing section 430, and the like in the above-described embodiment. The radiographic imaging system 1 in this case includes: the dynamic image capturing apparatus, which is constituted by the main body 10, the FPD 20, and the radiation irradiation apparatus 30; and the PC 5. With this configuration, a radiographic dynamic image captured by the dynamic image capturing apparatus is transmitted to the PC 5 via the main body 10. In the PC 5, processing of dynamic analysis of a radiographic dynamic image, processing of the above-described determination section, and the like are performed, and the processing results are transmitted to the PACS 2 and the like.

In addition, any of the embodiment described above is only illustration of an exemplary embodiment for carrying out the present invention, and the technical scope of the present invention shall not be construed limitedly thereby. That is, the present invention can be carried out in various forms without departing from the gist or the main features thereof.

Although embodiments of the present invention have been described and illustrated in detail, the disclosed embodiments are made for purposes of illustration and example only and not limitation. The scope of the present invention should be interpreted by terms of the appended claims.

1. A radiographic image processing apparatus that processes a dynamic image including a plurality of frames obtained by a dynamic image capturing apparatus related to radiographic imaging, the radiographic image processing apparatus comprising
a hardware processor that sets a region of interest in, among the plurality of frames of the dynamic image, a specific frame in which a predetermined status in a chest of a subject has been specified, wherein
the hardware processor determines information for categorizing a pathological condition in the region of interest based on first information and second information, the first information being information based on a signal value of the region of interest of the specific frame, the second information being information based on a temporal change amount of the signal value in the region of interest of the dynamic image.

2. The radiographic image processing apparatus according to claim 1, wherein
the hardware processor
determines, based on the first information, whether the pathological condition of the subject is classified into a first range or a second range, the first range being a range in which the signal value is within a predetermined range, the second range being a range in which the signal value is outside the predetermined range, and
determines, based on the second information, the information for categorizing the pathological condition in the region of interest within a range in which the pathological condition of the subject is classified.

3. The radiographic image processing apparatus according to claim 1, wherein
the hardware processor outputs the determined information for categorizing the pathological condition to a display medium.

4. The radiographic image processing apparatus according to claim 1, wherein
the specific frame is, among the plurality of frames, a frame in which the respiratory status of the subject has been specified.

5. The radiographic image processing apparatus according to claim 1, wherein
the dynamic image capturing apparatus is a movable medical cart.

6. The radiographic image processing apparatus according to claim 1, wherein
the hardware processor determines the information for categorizing the pathological condition based on a two-dimensional graph in which ranges of a plurality of the pathological conditions are segmented and which is constituted by a first axis and a second axis, the first axis indicating signal value information of the specific frame, the second axis indicating a temporal change amount of the signal value of the dynamic image as a whole.

7. A radiographic imaging system, comprising: a dynamic image capturing apparatus; and a radiographic image processing apparatus that processes a dynamic image including a plurality of frames obtained by the dynamic image capturing apparatus, wherein:
the radiographic image processing apparatus includes a hardware processor that sets a region of interest in, among the plurality of frames of the dynamic image, a specific frame in which a predetermined status in a chest of a subject has been specified, and
the hardware processor determines information for categorizing a pathological condition in the region of interest based on first information and second information, the first information being information based on a signal value of the region of interest of the specific frame, the second information being information based on a temporal change amount of the signal value in the region of interest of the dynamic image.

8. The radiographic imaging system according to claim 7, wherein
the dynamic image capturing apparatus is a movable medical cart.

9. A non-transitory computer-readable recording medium storing a program for a radiographic image processing apparatus to process a dynamic image including a plurality of frames acquired by a dynamic image capturing apparatus related to radiographic imaging, the program causing a computer to execute:
setting a region of interest in, among the plurality of images of the dynamic image, a specific frame in which a predetermined status in a chest of a subject has been specified; and
determining information for categorizing a pathological condition in the region of interest based on first information and second information, the first information being information based on a signal value of the region of interest of the specific frame, the second information being information based on a temporal change amount of the signal value in the region of interest of the dynamic image.
